(19) United States
(12) Patent Application Publication
(10) Pub. No.: US 2025/0302399 A1
(43) Pub. Date: Oct. 2, 2025

(54) CONTROL APPARATUS FOR MONITORING PATIENT ACTIVITY

(71) Applicant: Hill-Rom Services, Inc., Batesville, IN (US)

(72) Inventors: Robert Mark ZERHUSEN, Cincinnati, OH (US); Archana VAIDYANATHAN, Batesville, IN (US); Aditya SHINDE, Boston, MA (US); SaiAditya SANTHAKULLURU, Batesville, IN (US)

(21) Appl. No.: 19/092,730

(22) Filed: Mar. 27, 2025

Related U.S. Application Data
(60) Provisional application No. 63/571,674, filed on Mar. 29, 2024.

(51) Int. Cl. A61B 5/00

(52) U.S. Cl. CPC A61B 5/7435; A61B 5/7445; A61B 5/7475

(57) ABSTRACT

The present disclosure relates to systems and methods used to monitor patient activity and/or patient health status without accidentally affecting the operational activity of a patient support apparatus. The present disclosure relates to systems and methods of using one or more light indicators to identify different states of a patient monitoring device.

### CROSS-REFERENCE TO RELATED APPLICATIONS

This application claims priority under 35 U.S.C. § 119 (e) to U.S. Provisional Application No. 63/571,674, filed Mar. 29, 2024, which is expressly incorporated by reference herein.

### BACKGROUND

Various embodiments of this disclosure relate to a patient support apparatus, such as a hospital bed. More specifically, various embodiments of this disclosure are directed to monitor and identify patient activity and use of patient-watch or patient monitoring devices.

Recent advances in the medical field include improvements in systems and methods for monitoring patient activity. Assessing and tracking the activity and/or the health status of a patient is necessary to provide quality patient care. Systems and methods may be implemented to monitor a variety of health parameters, including but not limited to, pulse rate, blood pressure, blood oxygen level, posture of the patient, and other contingency events. Typically, systems implemented to monitor patient health and/or activity may include user interfaces that can be used by a user to provide inputs and/or control different operational parameters of the patient support apparatus.

However, any unintentional use of the user interface to enable or disable an operational activity may invoke a health-related risk for the patient. Therefore, there is a need to monitor patient activity and/or health status without accidentally affecting the operational activity of the patient support apparatus.

### SUMMARY

A medical bed control apparatus and method for controlling a patient monitoring activity or patient monitoring activity as shown in, and/or described in connection with, at least one of the figures, as set forth more completely in the claims.

In a first aspect, the present disclosure is directed to a patient monitoring system, comprising a patient support apparatus including an indicator, a controls system comprising a user interface (UI) including a display and a processor configured to change the display from a first display screen to a second display screen based on a first user input received on a first icon on the first display screen. The first icon corresponds to a patient monitoring device configured to monitor a patient activity. The second display screen is configured to indicate a first state of the patient monitoring device. The indicator is configured to exhibit a first color by illuminating at least a first light emitting diode (LED), and the first color indicates the first state of the patient monitoring device.

In some embodiments of the first aspect, the control system may be further configured to change the display from the second display screen to a third display screen based on a second user input received on a second icon on the second display screen. The second icon may correspond to a second state of the patient monitoring device, different from the first state of the patient monitoring device.

In some embodiments of the first aspect, the control system may be further configured to change the display from the third display screen to a fourth display screen based on a third user input received on a third icon on the third display screen. The third icon may correspond to a second state of the patient monitoring device, different from the first state of the patient monitoring device.

In some embodiments of the first aspect, the control system may be further configured to change the display from the fourth display screen to a fifth display screen based on a fourth user input received on a fourth icon on the fourth display screen. The fourth icon may correspond to a confirmation of the second state of the patient monitoring device.

In some embodiments of the first aspect, the control system may be further configured to change the state of the patient monitoring device from the first state to the second state if a user selects the fourth icon, and wherein the indicator is configured to exhibit a second color by illuminating at least a second LED. The second color may indicate the second state of the patient monitoring device.

In some embodiments of the first aspect, the first state and second state may be one of enabled and disabled.

In some embodiments of the first aspect, the indicator may comprise a plurality of light emitting diodes (LEDs), and each of the plurality of LEDs may have a different color.

In some embodiments of the first aspect, the processor may be further configured to control an intensity of light emitted from each of the plurality of LEDs.

In some embodiments of the first aspect, the first state and second state of the patient monitoring device may be indicated by blend of the light emitted from one or more of the plurality of LEDs.

In some embodiments of the first aspect, the one or more of the plurality of LEDs may partially illuminate an icon, corresponding to the first state or the second state of the patient monitoring device, on a surface in proximity to the patient support apparatus.

In some embodiments of the first aspect, the first user input, the second user input, the third user input, or the fourth user input may be an audio input.

In a second aspect, the current disclosure is directed to a control system, comprising a user interface (UI) including a display and a processor configured to change the display from a first display screen to a second display screen based on a first user input received on a first icon on the first display screen. The first icon may be configured to correspond to a patient monitoring device configured to monitor a patient activity (e.g., a patient-watch activity), wherein the second display screen is configured to indicate a first state of the patient monitoring device, wherein the control system is configured to illuminate a first indicator with a first color indicating the first state of the patient monitoring device.

In some embodiments of the second aspect, the control system may be further configured to change the display from the second display screen to a third display screen based on a second user input received on a second icon on the second display screen. The second icon may correspond to a second state of the patient monitoring device, different from the first state of the patient monitoring device.

In some embodiments of the second aspect, the control system may be further configured to change the display from the third display screen to a fourth display screen based on a third user input received on a third icon on the third display screen. The third icon may correspond to a second state of the patient monitoring device, different from the first state of the patient monitoring device.

In some embodiments of the second aspect, the control system may be further configured to change the display from the fourth display screen to a fifth display screen based on a fourth user input received on a fourth icon on the fourth display screen. The fourth icon may correspond to a confirmation of the second state of the patient monitoring device.

In some embodiments of the second aspect, the control system may be further configured to change the state of the patient monitoring device from the first state to the second state if a user selects the fourth icon, and wherein the control system is configured to illuminate a second indicator with a second color indicating the second state of the patient monitoring device.

In some embodiments of the second aspect, the first state and second state may be one of enabled and disabled.

In some embodiments of the second aspect, the first or second indicator may comprise a plurality of light emitting diodes (LEDs), and each of the plurality of LEDs has a different color.

In some embodiments of the second aspect, the processor may be further configured to control an intensity of light emitted from each of the plurality of LEDs.

In some embodiments of the second aspect, the first state and second state of the patient monitoring device may be indicated by blend of the light emitted from one or more of the plurality of LEDs.

In some embodiments of the second aspect, the one or more of the plurality of LEDs may partially illuminate an icon, corresponding to the first state or the second state of the patient monitoring device, on a surface in proximity to the patient support apparatus.

In some embodiments of the second aspect, the first user input, the second user input, the third user input, or the fourth user input may be an audio input.

In a third aspect, the present disclosure is directed to a patient monitoring system, a patient support apparatus including an indicator, wherein the indicator is configured to indicate a first state of a patient monitoring device by illuminating at least a first light indicator and exhibiting a first color, and wherein the first color indicates a first state of the patient monitoring device.

In some embodiments of the third aspect, the indicator is further configured to indicate a second state of a patient monitoring device by illuminating at least a second light indicator, wherein the second color indicates a second state of the patient monitoring device.

In some embodiments of the third aspect, the first state and the second state are one of enabled and disabled.

In some embodiments of the third aspect, wherein the first state and the second state are one of enabled and disabled.

In some embodiments of the third aspect, wherein the first or second light indicator comprises a plurality of light emitting diodes (LEDs), and each of the plurality of LEDs has a different color.

In some embodiments of the third aspect, wherein the processor is further configured to control an intensity of light emitted from each of the plurality of LEDs.

In some embodiments of the third aspect, wherein the first state and second state of the patient monitoring device is indicated by blend of the light emitted from one or more of the plurality of LEDs.

In some embodiments of the third aspect, wherein the one or more of the plurality of LEDs partially illuminates an icon, corresponding to the first state or the second state of the patient monitoring device, on a surface in proximity to a patient support apparatus.

These and other features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

FIG. 1 is a block diagram that illustrates an exemplary network environment including a patient support apparatus and a control system to control and monitor patient activity and/or patient monitoring devices, according to an embodiment of the present disclosure.

FIG. 2A illustrates a first user interface display screen for controlling a state of the patient monitoring device, according to a first embodiment of the present disclosure.

FIG. 2B illustrates a second user interface display screen for controlling the state of a disabled patient monitoring device, according to a first embodiment of the present disclosure.

FIG. 2C illustrates a fourth user interface display screen for controlling the state of a disabled patient monitoring device, according to a first embodiment of the present disclosure.

FIG. 2D illustrates a sixth user interface display screen for controlling the state of a disabled patient monitoring device, according to a first embodiment of the present disclosure.

FIG. 2E illustrates an eight user interface display screen for controlling the state of a disabled patient monitoring device, according to a first embodiment of the present disclosure.

FIG. 2F illustrates a third user interface display screen for controlling the state of an enabled patient monitoring device, according to a first embodiment of the present disclosure.

FIG. 2G illustrates a fifth user interface display screen for controlling the state of an enabled patient monitoring device, according to a first embodiment of the present disclosure.

FIG. 2H illustrates a seventh user interface display screen for controlling the state of an enabled patient monitoring device, according to a first embodiment of the present disclosure.

FIG. 2I illustrates a ninth user interface display screen for controlling the state of an enabled patient monitoring device, according to a first embodiment of the present disclosure.

FIG. 3 is a flowchart that illustrates operations of a first method for controlling the state of a patient monitoring device, according to an embodiment of the present disclosure.

FIG. 4A illustrates a first user interface display screen for controlling a state of the patient monitoring device, according to a second embodiment of the present disclosure.

FIG. 4B illustrates a second user interface display screen for controlling the state of the patient monitoring device, according to a second embodiment of the present disclosure.

FIG. 4C illustrates a third user interface display screen for controlling the state of the patient monitoring device, according to a second embodiment of the present disclosure.

FIG. 4D illustrates a fourth user interface display screen for controlling the state of a disabled patient monitoring device, according to a second embodiment of the present disclosure.

FIG. 4E illustrates a sixth user interface display screen for controlling the state of a disabled patient monitoring device, according to a second embodiment of the present disclosure.

FIG. 4F illustrates an eight user interface display screen for controlling the state of a disabled patient monitoring device, according to a second embodiment of the present disclosure.

FIG. 4G illustrates a fifth user interface display screen for controlling the state of an enabled patient monitoring device, according to a second embodiment of the present disclosure.

FIG. 4H illustrates a seventh user interface display screen for controlling the state of an enabled patient monitoring device, according to a second embodiment of the present disclosure.

FIG. 4I illustrates a ninth user interface display screen for controlling the state of an enabled patient monitoring device, according to a second embodiment of the present disclosure.

FIG. 5 is a flowchart that illustrates operations of a second method for controlling the state of a patient monitoring activity, according to a second embodiment of the present disclosure.

FIG. 6A illustrates an indicator on the patient support apparatus exhibiting a white light to indicate an operating control system but a disabled patient monitoring device (e.g., incontinence pad).

FIG. 6B illustrates an indicator on the patient support apparatus exhibiting a green light to indicate an operating control system and an enabled patient monitoring device (e.g., incontinence pad).

FIG. 6C illustrates an indicator on the patient support apparatus exhibiting a white light to indicate an operating control system and a third state of the patient monitoring device, a wet incontinence pad.

FIG. 6D illustrates an indicator on the patient support apparatus exhibiting a yellow light formed by the illumination of more than one LED to indicate a disabled patient monitoring device (e.g., incontinence pad).

### DETAILED DESCRIPTION

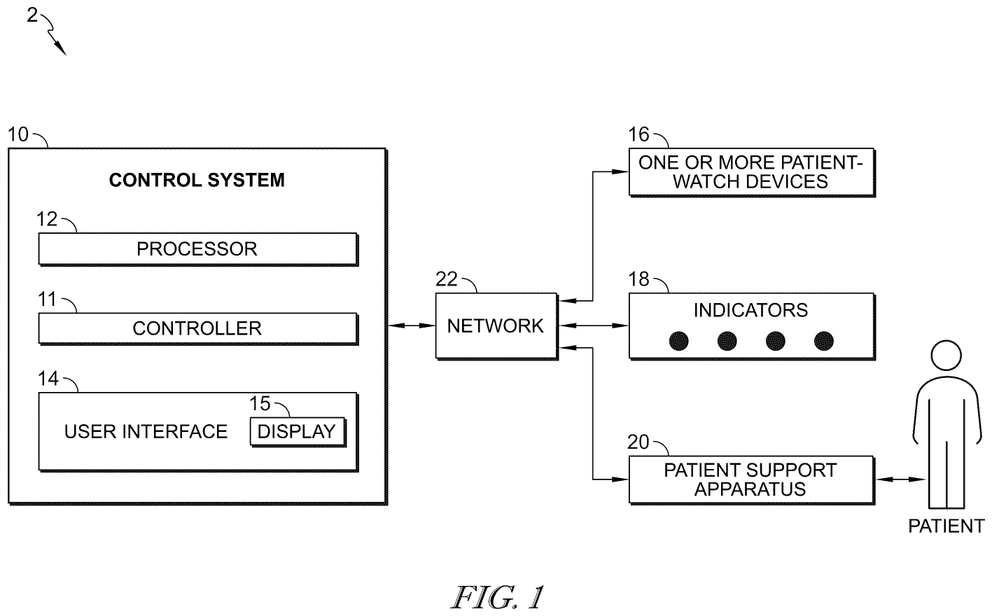

FIG. 1 is a block diagram that illustrates an exemplary embodiment of a patient monitoring system 2, comprising a patient support apparatus 20 and a control system or a control apparatus 10. The patient support apparatus 20 is in communication with the control system 10 that includes a controller 11, a processor 12, and a user interface 14. The control system 10 is configured to be in communication with one or more patient monitoring devices 16 and one or more indicators 18. The control system 10 may be located on the patient support apparatus 20 or may be positioned separately from the patient support apparatus 20. The control system 10 may be communicatively coupled to one or more patient monitoring devices 16, one or more indicators 18, and a communication device of the patient support apparatus 20, via a network 22.

The control system 10 may include suitable logic, circuitry, interfaces, and/or code that may be configured to control and/or monitor one or more patient activities of a patient positioned on the patient support apparatus 20. The patient activities can be monitored and/or controlled by utilizing one or more of the patient monitoring devices 16 (i.e., a device to monitor health, physical condition, or activity of the patient). A user and/or caregiver can use the user interface 14 to monitor and/or control patient activities. In some embodiments, the patient monitoring devices 16 may be located in the control system 10. In other embodiments, the control system 10 may be communicably coupled with the patient monitoring devices 16 via the network 22.

The processor 12 may comprise suitable logic, circuitry, and interfaces that may be configured to perform various arithmetic processing and control operations associated with the control system 10 based in the input received through the user interface 14. The processor 12 may include one or more processing units that may be implemented as separate processors.

The user interface 14 may comprise suitable logic, circuitry, and interfaces that may be configured to display the one or more states of one or more patient activities based on information received from the one or more patient monitoring devices 16. The user interface 14 includes a display 15 that can show the information received from the one or more patient monitoring devices 16. The user interface 14 may comprise at least one of several known technologies such as, but not limited to, Liquid Crystal Display (LCD) display, a Light Emitting Diode (LED) display, a plasma display, or an Organic LED (OLED) display technology, or other display devices.

Each of the one or more patient monitoring devices 16 may comprise suitable logic, circuitry, and interfaces that may be configured to monitor at least one condition (health or physical) associated with the patient positioned on the patient support apparatus 20. The patient monitoring device 16 may be a heart-rate monitor that monitors a heart-rate of the patient, an oxygen-level monitor that monitors an oxygen-level in the blood of the patient, an incontinence detection device that monitors an occurrence of an incontinence event, or a similar monitoring device.

In one embodiment, one or more of the patient monitoring devices 16 may be an image capturing device (like a camera) that can monitor the physical condition of the patient. The one or more patient monitoring devices 16 may be located on the control system 10 or may be communicably coupled to the control system 10 via the network 22. In one embodiment, the one or more patient monitoring devices 16 may be integrated with the patient support apparatus 20 to monitor the health or physical conditions of the patient positioned on the patient support apparatus 20.

The one or more indicators 18 may comprise suitable logic, circuitry, and interfaces that may be configured to indicate different states (e.g., enabled state or disabled state) of the one or more patient monitoring devices 16 or the status of one or more the patient activities An indicator may include a light-emitting indicator, an audio indicator, and/or a projector. The light emitting indicator may include one or more light emitting diodes (LEDs) or similar technology. In a case, where a plurality of LEDs are included in the light indictor, each of the plurality of LEDs may emit light of different colors. In an embodiment, the one or more indicators 18 may be included on the control system 10. In another embodiment, one or more indicators 18 may be integrated with the patient support apparatus 20 and may controlled by the control system 10, via the network 22.

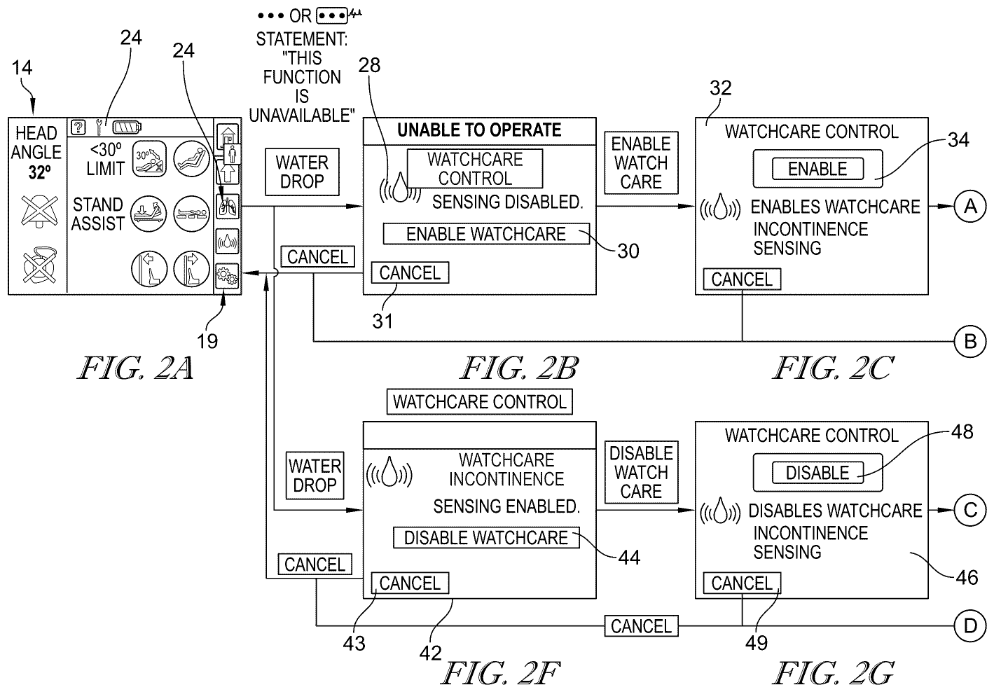

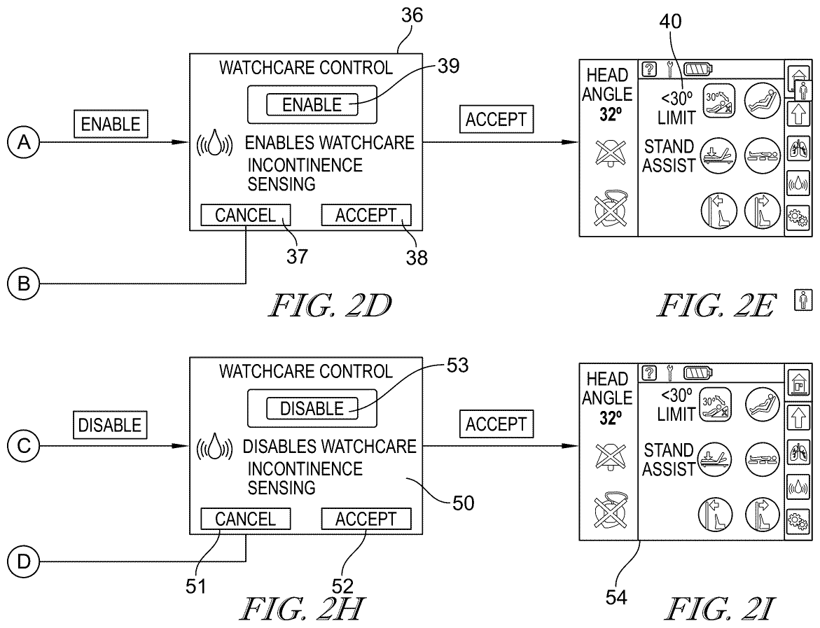

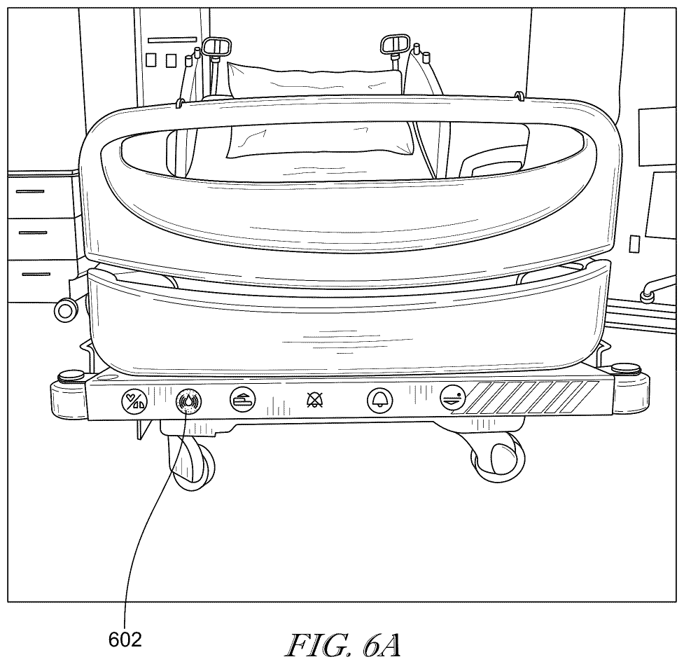

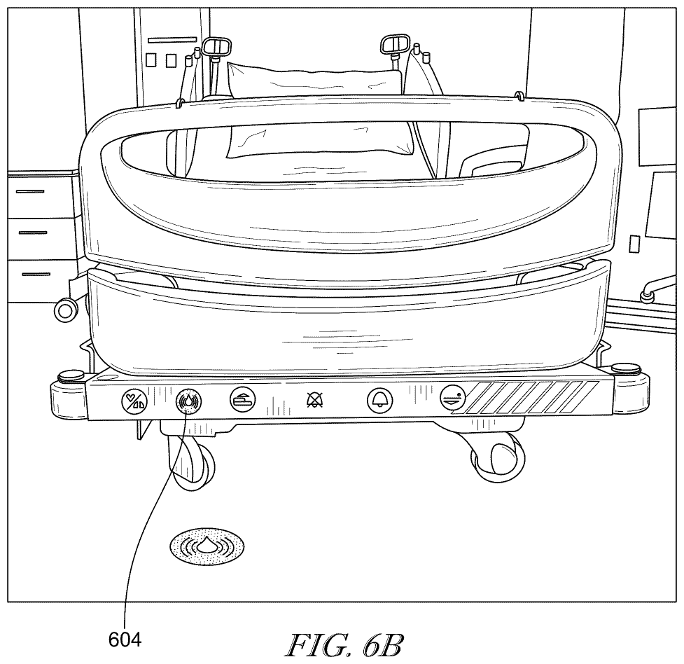

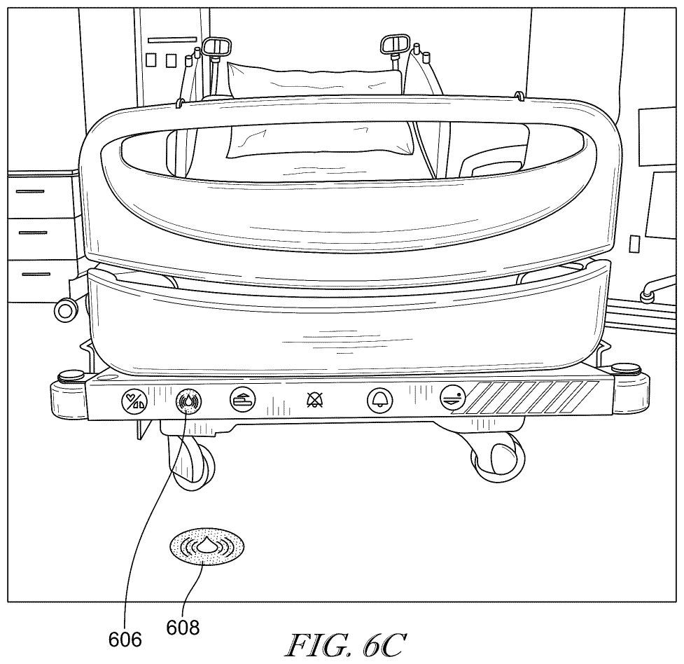

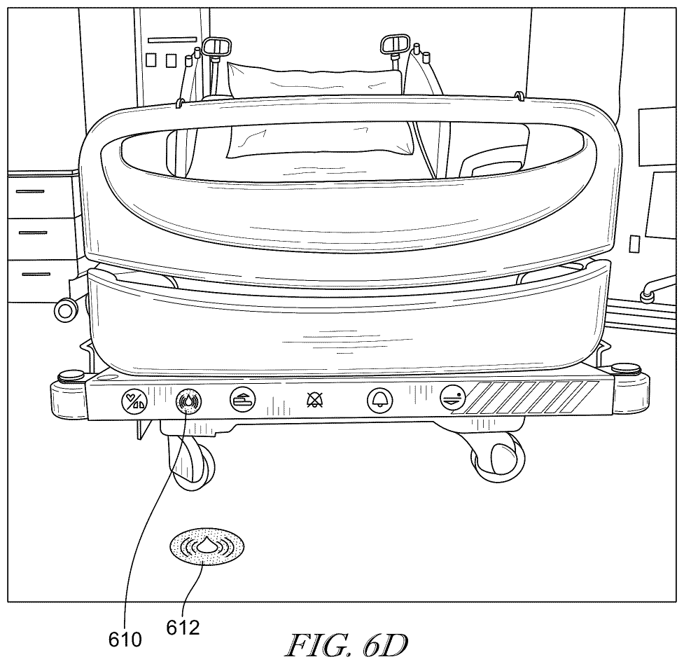

As shown in FIGS. 2-5, the user interface 14 of the control system 10 may include prompts or icons that the caregiver or user can utilize to control and/or monitor the patient activities based on the information obtained from the one or more patient monitoring devices 16. As shown in FIGS. 6A-6D, the control system 10 may utilize one or more indicators 18 to indicate the recent status of the patient activity and/or the state of the more patient monitoring devices 16.

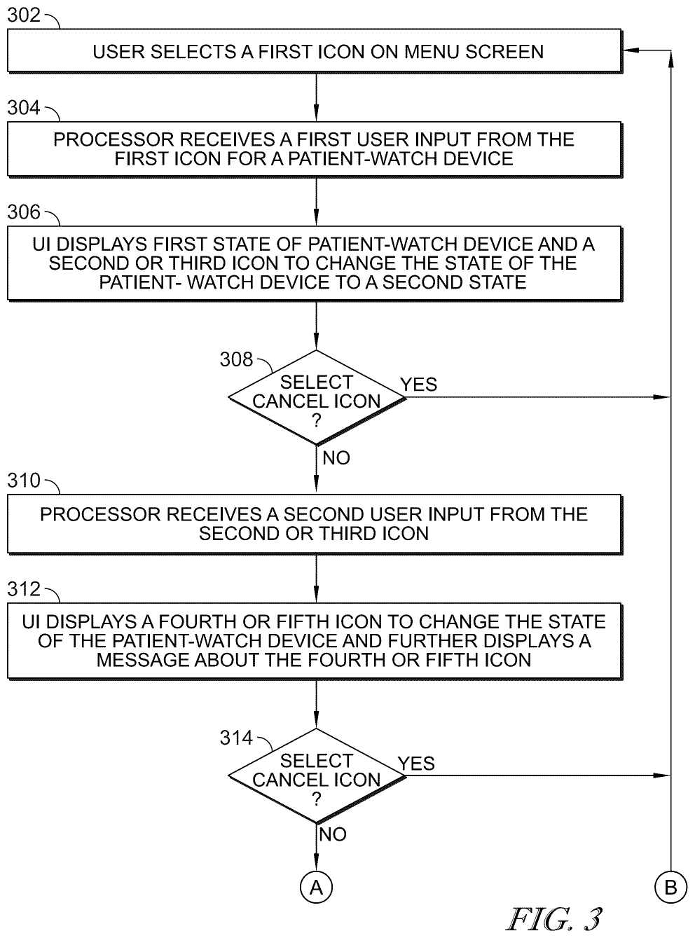

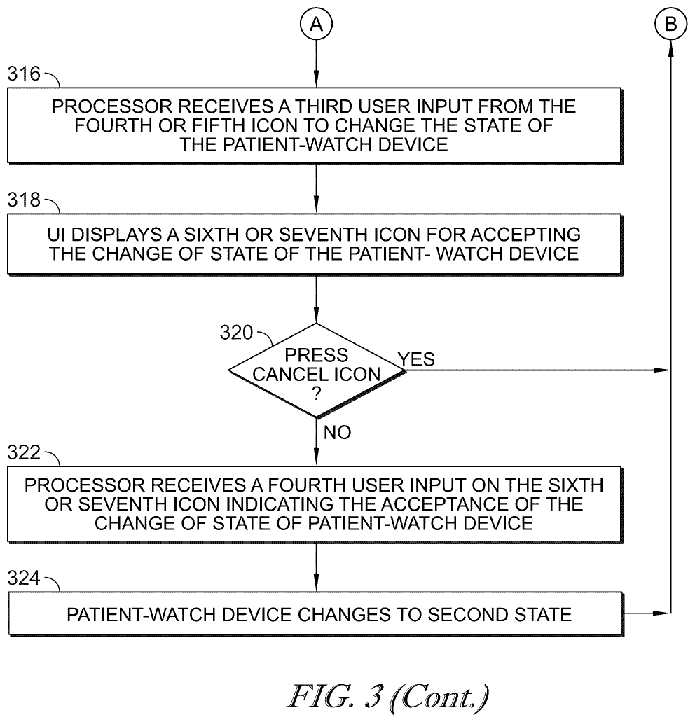

FIGS. 2A-2I illustrate an exemplary user interface 14. The user interface 14 includes a first display screen 24 and a menu screen 19. When the user selects an icon or prompt on the menu screen 19, a workflow is initiated as shown in FIGS. 2A-2I. FIG. 3 is a flowchart an enabled state. The sequence of display screen 24, 42, 46, 50, 54 in FIGS. 2A, 2F-2I illustrate the transition of the patient monitoring device 16 (for example incontinence detection device) from the enabled state to the disabled state.

The user selects a first icon 26 on the menu screen 19 (FIG. 2A) as also indicated in step 302 of FIG. 3. The first icon 26 corresponds to the patient monitoring device 16 directed to a first patient activity (for example incontinence detection for the patient). The processor 12 is configured to receive a first user input when the user selects the first icon 26 as shown in step 304 of FIG. 3. A second display screen 28 or a third display screen 42 appears on the user interface 14, as shown in FIGS. 2B and 2F after the first input is received. The first user input may correspond to a click or touch on the first icon 26 by the user on order to change the state of the patient monitoring device 16 (either from enabled to disabled or vice versa). As illustrated in FIG. 2B and as shown in step 306 of FIG. 3, the processor 12 is configured to change the first display screen 24 to the second display screen 28 if the patient monitoring device 16 is currently disabled. Alternatively, as illustrated in FIG. 2F and as shown in step 306 of FIG. 3, the processor 12 is configured to change the first display screen 24 to the third display screen 42 if the patient monitoring device 16 is currently enabled (FIG. 2F).

The second and third display screens 28, 42 may display a message indicating a current state (as a first state) of the patient monitoring device 16. The second display screen 28 and the third display screen 42 are configured to show a second icon 30 and a third icon 44 respectively. Each of the second icon 30 and the third icon 44 can be selected to change the patient monitoring device 16 from the first state to an alternate state (second state). As indicated in step 306 of FIG. 3, the user interface 14 displays the first state of the patient monitoring device 16 and shows an icon 30, 44 that can change the state of the patient monitoring device 16 from the first state to the second state.

The second and third display screens 28, 42 may further display a cancel icon 31, 43 that is configured to revert the user interface 14 back to the first display screen 24. For example, if the cancel icon 31, 43 of the screen second and third display screens 28, 42 is selected as indicated in step 308 of FIG. 3, the user interface 14 may transition to the first display screen 24.

In some embodiments, the current state (first state) may be the disabled state and the alternate state may be the enabled state. For example, the second display screen 28 of FIG. 2B may indicate that the patient activity is not being monitored indicating that the corresponding patient monitoring device 16 may be currently disabled. In case of incontinence detection activity for the patient, the incontinence device (as patient monitoring device 16) may be disabled and may not be monitoring the incontinence activity of the patient. This is shown in the second display screen 28 of FIG. 2B as “Incontinence sensing disabled”. In another embodiment, as shown in the third display screen 42 of FIG. 2F, the current state (first state) may be the enabled state and the alternate state (second state) may be the disabled state. For example, the third display screen 42 of FIG. 2F may indicate that the patient activity is being monitored indicating the corresponding patient monitoring device 16 may be currently enabled on the patient support apparatus 20.

The processor 12 is configured to receive a second user input from the second or third icon 30, 44 on the second or third display screens 28, 42 as indicated in step 310 of FIG. 3. The second or third icon 30, 44 may correspond to the second state of the patient activity that may be different from the first state (current state) of the patient monitoring device 16. For example, as shown on the second display screen 28, the second state may correspond to the enabled state to allow the patient monitoring device 16 to operate and monitor the corresponding patient activity. Further, as shown on the third display screens 42, the second state may correspond to the disabled state to disable the patient monitoring device 16.

The processor 12 may be configured to change the user interface 14 from showing the second or third display screens 28, 42 to a fourth or fifth display screen 32, 46 as shown in FIGS. 2C and 2G respectively. This change to the fourth or fifth display screen 32, 46 may be based on the receipt of the second user input from the second or third icon 30, 44. For example, the second or third icon 30, 44 may be used when transitioning to the alternate state as indicated in step 310 of FIG. 3.

The fourth or fifth display screen 32, 46 may display a fourth or fifth icon 34, 48 which may indicate the alternate state or the second state of the patient monitoring device 16. The fourth or fifth icon 34, 48 may allow the user to confirm the change from the first state to the second state and avoid any accidental or inadvertent change of the operational states of the patient monitoring device 16. This may further avoid any accident or health-related issues that may arise (for example skin related issues or skin breakdown/damage due to the incontinence activity). Such issues may arise due to any accidental change in the state (like disabling) of the patient activities triggered by the user from the conventional user interfaces.

The processor 12 may be further configured to receive a user input from a second cancel icon 35, 49 on the fourth or fifth display screen 32, 46 respectively. Based on user input from the second cancel icon 35, 49, the processor 12 may further cause the user interface 14 to revert back to display the first display screen 24. For example, when the second cancel icon 35, 49 on the fourth or fifth display screen 32, 46 is selected, the user interface 14 transitions to the first display screen 24 as indicated in step 314 of FIG. 3.

When the processor 12 receives a third user input from the fourth or fifth icon 34, 48 on the fourth or fifth display screens 32, 46 as indicated in step 316 of FIG. 3, the processor 12 is configured to change the display from the fourth or fifth display screens 32, 46 to a sixth or seventh display screen 36, 50 (FIGS. 2D and 2H). For example, when the fourth or fifth icon 34, 48 is pressed, the user interface 14 transitions to the sixth or seventh display screen 36, 50.

The sixth or seventh display screens 36, 50 may display a sixth or seventh icon 38, 52 that may allow the user to “accept” the selection of the alternate or second state of the patient monitoring device 16, as also indicated in step 318 of FIG. 3. The “accept” icons 38, 52 may be used to the user to confirm the selection of the alternate state for the patient monitoring device 16 or to receive confirmation for the changes of states. The sixth and seventh display screens 36, 50 may also include icons 39, 53 with an “Enable” or “Disable” label. In some embodiments, the icons 39, 53 with an “Enable” or “Disable” label may be used to confirm the selection of the alternate state for the patient monitoring device 16 or to receive confirmation for the changes of states. The sixth or seventh display screens 36, 50 may further include a message (like “Enable Watchcare® incontinence sensing”) describing the performance of the accept icons 38, 52.

The sixth or seventh display screens 36, 50 may further include cancel icons 37, 51. Selection of the cancel icons 37, 51 may cause the processor 12 to change the display to the first display screen 24 (as shown in step 320 of FIG. 3). In some embodiments, based on the user interface 14, the transition from the fourth or fifth display screens 32, 46 to the sixth or seventh display screens 36, 50, may change the color of the icons 34, 48 and 39, 53. For example, the icons 34, 48 may be blue and the icons 39, 53 may be green. The difference in color may indicate the impending final selection of the alternate state of the patient monitoring device 16.

The processor 12 may be configured to receive a fourth user input from the accept icons 38, 52 on the sixth or seventh display screens 36, 50. Selection of the accept icons 38, 52 may correspond to an acceptance by the user to change the patient monitoring device 16 from the current state to the alternate state, as indicated in step 322 of FIG. 3. The patient monitoring device 16 may change from the current state to the alternate state as indicated in step 324 of FIG. 3. Subsequently, the user interface 14 may further transition from the sixth or seventh display screens 36, 50 to an eighth or ninth display screen 40, 54 (FIGS. 2E and 2I). In some embodiments, at least one of the first user input, the second user input, the third user input, and/or the fourth user input may be an audio input (i.e., voice command), rather than a touch, click, or press input.

Therefore, the sequential execution or traversal from the first screen using multiple user inputs, to change the state of the patient monitoring device 16 may avoid any accidental change of state that may impact the health of the patient. For example, any accidental disablement of the incontinence detection may impact the health (e.g., skin breakdown) of the patient if the incontinence related to the patient is ignored by the caregiver after the accidental disablement of the incontinence detection device. Thus, the disclosed control system 10 may mitigate the risk related to the health issue of the patient which may be caused due to any accidental change of state of the patient monitoring devices 16 used to monitor patient activities.

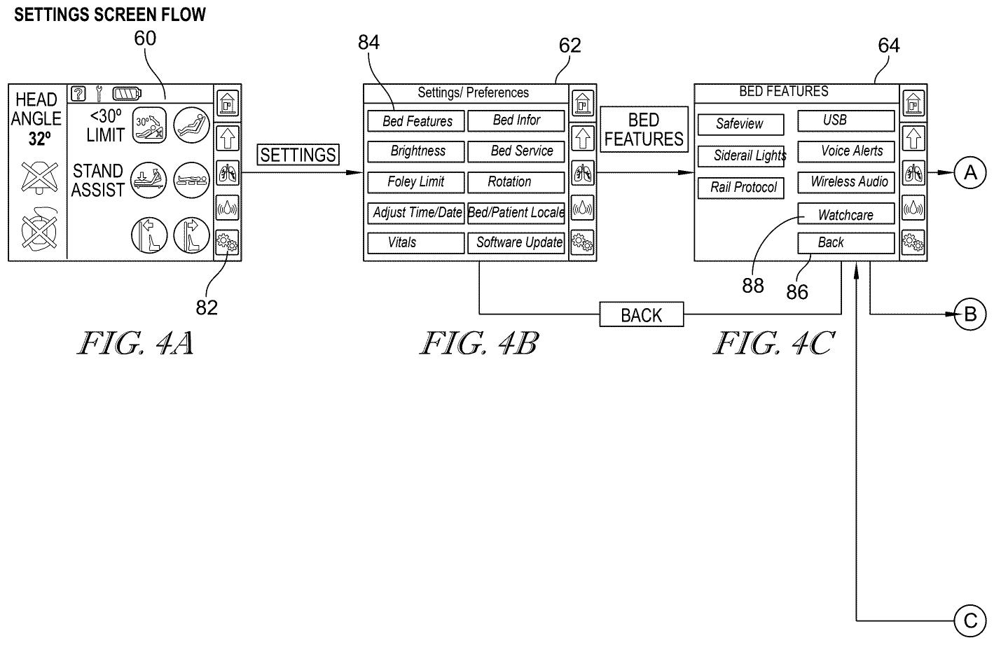

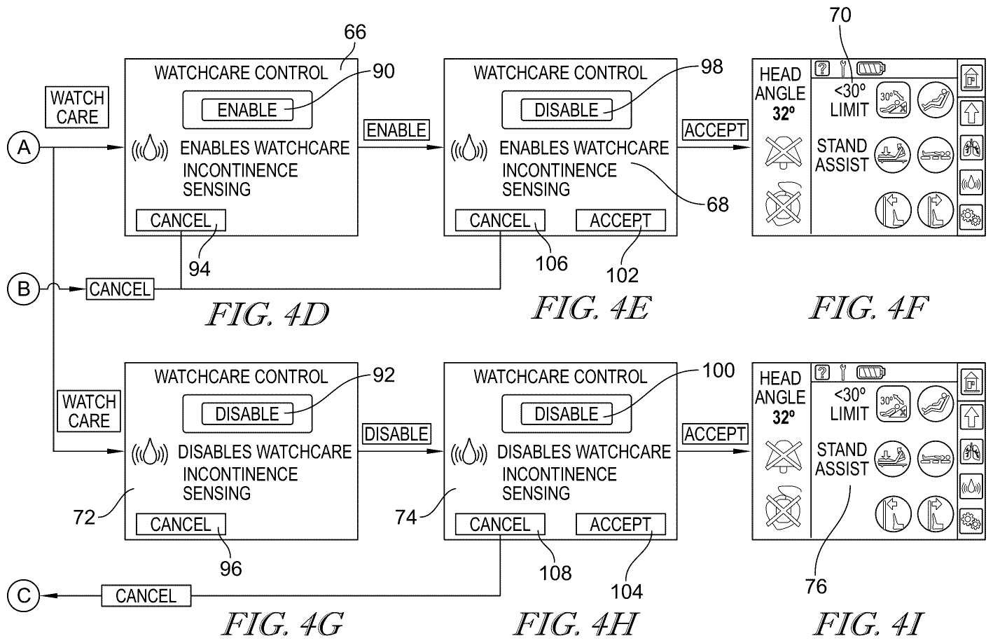

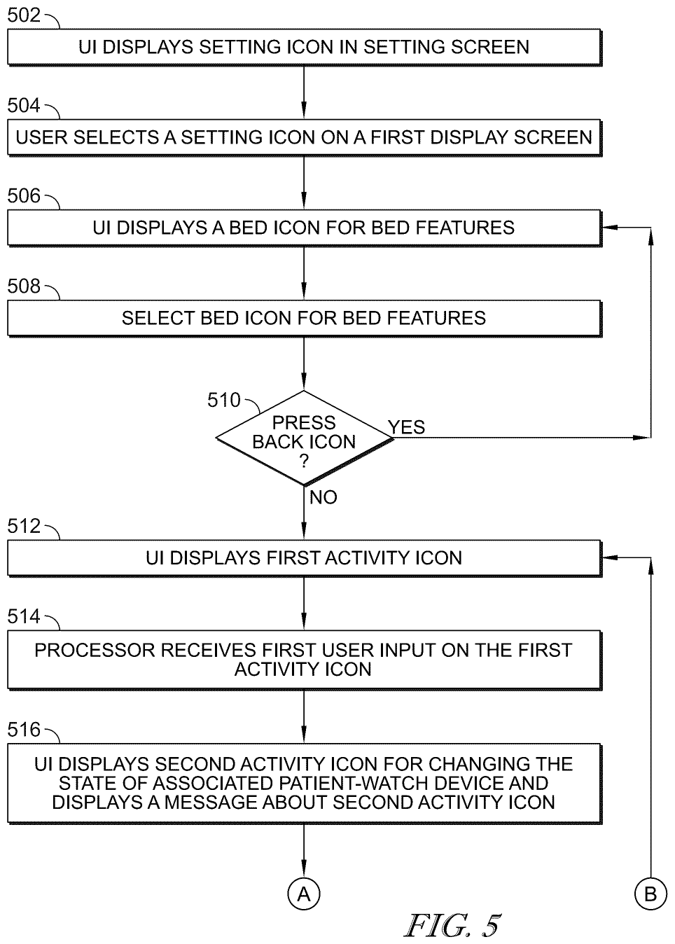

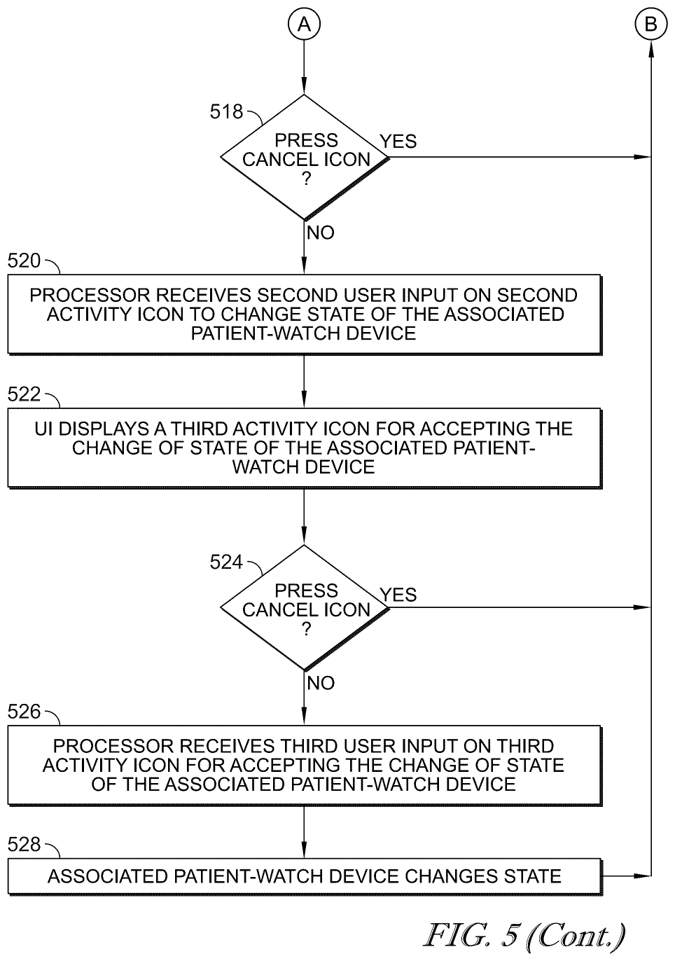

FIGS. 4A-4I is a diagram that illustrates a second exemplary scenario for a user interface 14 for controlling a state of the patient monitoring device 16. As shown in FIGS. 4A, the user interface 14 may include a first display screen 60 that may be a default screen of the user interface (UI). FIGS. 4A-4I illustrate a workflow which may be initiated from the default display screen 60. FIG. 5 is a flowchart that illustrates operations of the workflow shown in FIGS. 4A-4I. The sequence of display screens 60, 62, 64, 66, 68, 70 illustrates the transition of the patient monitoring device 16 from the disabled state to the enabled state. The sequence of display screens 60, 62, 64, 72, 74, 76 illustrates the transition of the patient monitoring device 16 from the enabled state to the disabled state.

As shown in FIG. 4A and indicated in step 502 of FIG. 5, the first display screen includes a setting icon 82. When the setting icon 82 on the first display screen 60 is selected by the user, the user interface 14 may transition from the first display screen 60 to a second display screen 62. The user interface 14 may receive a first user input (i.e., click or touch) from the setting icon 82 (as indicated in step 504 of FIG. 5) based on which the processor 12 may change the user interface 14 display from the first display screen 60 to the second display screen 62.

As shown in FIG. 4B and indicated in step 506 of FIG. 5, the second display screen 62 may display icons for various features or settings related to the patient support apparatus 20, related to one or more patient monitoring devices 16, and/or related to the patient health monitoring, such as, but not limited to, bed features, bed information, patient's vitals, display settings (brightness), and software update. If a bed icon 84 for the bed features on the second display screen 62 is selected, the second display screen 62 may change to a third display screen 64 (FIG. 4C). The user interface 14 may receive a second user input from on the bed icon 84 as indicated in step 508 of FIG. 5. Upon receipt of the second user input, the processor 12 may change the second display screen 62 to the third display screen 64.

As indicated in step 510 of FIG. 5, if a back icon 86 on the third display screen 64 is selected, the user interface 14 may transition back to the second display screen 62 instead of progressing to a fourth display screen 66. The third display screen 64 may further display different icons for various bed features, such as, but not limited to, rail protocol, siderail lights, and various activity icons for monitoring patient activity on the bed as shown in FIG. 4C and as also indicated in step 512 of FIG. 5. An icon for monitoring a first patient activity, referred to as a first activity icon 88, is illustrated on the third display screen 64. The processor 12 may be configured to receive a first user input form the first activity icon 88 on the third display screen 64 as shown in step 514 of FIG. 5. The first user input may correspond to a click or touch on the first activity icon 88 by a caregiver or a user to change the state of the patient monitoring device 16 corresponding to the first activity icon. The patient monitoring device 16 may be associated with patient support apparatus 20 (e.g., bed 20). The processor 12 may be further configured to change the third display screen 64 to a fourth or fifth display screen 66, 72 based on the receipt of the first user input on the first activity icon 88 and the status of the patient monitoring device 16 corresponding to the first activity icon.

As shown in in FIGS. 4D and 4G and indicated in step 516 of FIG. 5, the fourth or fifth display screen 66, 72 may include a second activity icon 90 or 92 for changing the state of the patient monitoring device 16 from the current state (i.e., first state) to the alternate state (i.e., second state). The fourth or fifth display screen 66, 72 may further display a message (like “Enable Watchcare® incontinence sensing”) describing the function of the second activity icon 90, 92. The fourth or fifth display screen 66, 72 may further include a cancel icon 94, 96 to transition back to the third display screen 64. In some embodiments, as shown in FIG. 4G, the current state may be the enabled state and the alternate state may be the disabled state. In other embodiments, as shown in FIG. 4D, the current state may be the disabled state and the alternate state may be the enabled state.

As indicated in step 518 of FIG. 5, if the cancel icon 94, 96 of the fourth or fifth display screen 66, 72 is selected, the display may transition back to the third display screen 64. Alternatively, as indicated in step 520 of FIG. 5, the processor 12 may be configured to receive a second user input from the second activity icon 90, 92 on the fourth or fifth display screen 66, 72. As shown in FIGS. 4E and 4H, the processor may be configured to change the fourth or fifth display screen 66, 72 to a sixth or seventh display screen 68, 74 based on the receipt of the second user input.

As shown in FIGS. 4E and 4H, the user interface 14 may display a selection label 98, 100 indicating the selection of the alternate/second state of the patient monitoring device 16. For example, the sixth display screen 68 may show a “Enable” label. The seventh display screen 74 may show a “Disable” label. As indicated in step 522 of FIG. 5, the sixth or seventh display screen 68, 74 may further include a third activity icon 102, 104 (e.g., an accept icon) to confirm the selection of the alternate state for the patient monitoring device 16. The sixth or seventh display screen 68, 74 may further display a message such as “Enable Watchcare® incontinence sensing” or like “Disable Watchcare® incontinence sensing” describing the transition in state. As indicated in step 524 of FIG. 5, the sixth or seventh display screen 68, 74 may further include a cancel button 106, 108, the use of which would return the user interface 14 to the fourth or fifth display screen 66, 72. In some embodiments, when the user interface 14 transition from the fourth or fifth display screen 66, 72 to the sixth or seventh display screen 68 74, the color of the icon to indicate selection (e.g., selection label 98, 100) may change. For example, as shown in in FIGS. 4E and 4H, the color may change from blue to green.

As indicated in step 526 of FIG. 5, the processor 12 may be further configured to receive a third user input from the third activity icon 102, 104 that may correspond to an acceptance from the user to change the patient monitoring device 16 from the current state to the alternate state. As indicated in step 528 of FIG. 5, the processor 12 may be further configured to change the patient monitoring device 16 from the first state to the second state based on the receipt of the third user input. Subsequently, the user interface 14 may transition from the sixth or seventh display screen 68 74 to an eighth or ninth display screen 70, 76 illustrated in FIGS. 4F and 4I. In some embodiments, the eighth or ninth display screen 70, 76 may be the default screen or the first display screen 60 shown in FIG. 4A. Therefore, as illustrated, the sequential execution or traversal using multiple user inputs, to change the states of the patient activity may further avoid accidental change of the states that may impact the health of the patient.

In some embodiments, as shown in FIG. 1, the control system 10 may further include one or more indicators 18. In some embodiments, the control system 10 may be communicably coupled to the patient support apparatus 20, via the network 22 and the indicators 18 may be positioned on the patient support apparatus 20. The one or more indicators 18 may include light indicators and may be positioned at different places of the patient support apparatus 20. For example, the indicators 18 may be positioned on an outer surface of the patient support apparatus 20 to project light or image at a foot end of the patient support apparatus 20. The light indicators may include at least one indicator corresponding to each of the one or more patient monitoring devices 16. The indicator 18 for a particular patient monitoring device 16 may indicate the current state of the patient monitoring device 16 to the health operator, like whether the incontinence detection device is at the enabled state (active) or disabled state to monitor the incontinence activity of the patient on the patient support apparatus 20. Each patient monitoring device 16 may have one, two, or more than two states. Each state of the patient monitoring device 16 may indicate different patient activity and may be exhibited by one or more indicators 18.

In some embodiments, at least one indicator 18 corresponding to the patient monitoring device may include a plurality of LEDs (light emitting diodes), where each LED may correspond to a particular or different color. Therefore, the indicator 18 may emit light one or more colors to indicate different states of the patient monitoring device.

FIGS. 6A-6D illustrate exemplary scenarios for indicators in a patient support apparatus 20 to indicate different states of patient monitoring device 16, according to the present disclosure. Referring to FIGS. 6A-6D, the patient support apparatus 20 may include one or more indicators 18 each including a plurality of LEDs. Each LED may illuminate different color to show different states of the patient monitoring device 16 or patient activity. In an embodiment, each of the plurality of LEDs partially illuminates an icon, corresponding to the patient monitoring device 16. As shown in FIG. 6A, an LED indicator 602 illuminates white color when the patient monitoring device 16 is an incontinence detection device, but the incontinence detection device does not detect a smart incontinence pad. As shown in FIG. 6B, an LED indicator 604 illuminates green color when the incontinence detection device detects the smart incontinence pad. As shown in FIG. 6C, an LED indicator 606 illuminates a flashing amber color when the incontinence detection device detects that the smart incontinence pad of the patient is wet and needs to be changed. In an embodiment, the LED indicator 606 illuminates an icon, corresponding to the patient activity or patient monitoring device 16, on a surface (like floor) which is in proximity to the patient support apparatus 20. For example, The LED indicator 606 may project an image 608 of the flashing amber colored on the floor when the incontinence detection device detects that the smart incontinence pad is wet and needs to be changed. Thus, the LED indicator 606 emits flashing amber color to indicate an alerting state of the incontinence detection device.

The control of the states of the patient activity (for example incontinence detection device) using the user interface 14 of the control system 10 is described in detail, for example, in FIGS. 2-5. The LED indicator 604 may emit green color to indicate an activated or enabled state of the incontinence detection device. The LED indicator 604 may emit white color to indicate a standby or disabled state of the incontinence detection device. The LED indicator 606 emits flashing amber color to indicate an alerting state of the incontinence detection device. The projected image 608 or icon on the floor may alert the health professional about the current state of the patient activity or the corresponding patient monitoring device so that the health professional may take appropriate measures for the patient based on the current state of the activity. For example, in case the patient monitoring device 16 monitoring is disabled, then the health professional may manually check the status for the patient. For example, in case the incontinence pad is wet, and the corresponding patient monitoring device is disabled, then the LED indicator may alert a nurse to check the pad regularly and change the pad to avoid any skin-related breakdown with the patient. Therefore, the projection of the current state of the patient activity or patient monitoring device, via the LED indicator using different colors may help the health professional to avoid any medical risk related to the patient.

In an embodiment, as shown in FIG. 6D, an LED indicator 610 may emit yellow light to indicate a disabled state of the incontinence detection device. In an embodiment, the disabled state of the patient activity is indicated by blend of the light emitted from each of the plurality of LEDs of the LED indicator. For example, the LED indicator 610 may emit yellow light by blending lights from multiple LEDs. For example, the LED indicator 610 may emit yellow light by blending lights from a green color LED and an amber color LED of the plurality of LEDs. In an embodiment, the LED indicator 610 may illuminate using the green LED and the amber LED together to indicate the disabled state of the incontinence detection device.

In an embodiment, the processor 12 or the processor may control the brightness or intensity of each of the plurality of LEDs to illuminate the image/icon with a specific color corresponding to a specific state of the patient monitoring device 16 (for example incontinence detection device). In an embodiment, the processor 12 may control the brightness or intensity of each LED to illuminate the image/icon with blending of multiple colors to further indicate a specific state of the patient monitoring device 16.

In an embodiment, as shown in FIG. 6D, the LED indicator 610 may project an image 612 of the illuminated icon on the floor. For example, the LED indicator 610 may project an image of the icon of blended colors on the floor to indicate the disabled state of the incontinence detection device/activity. In another embodiment, the LED indicator 610 may continuously flash an image/icon of blended colors on the floor to indicate the disabled state of the patient activity or patient monitoring device 16 (for example incontinence detection device).

In some embodiments, to reduce the number of LEDs, the patient monitoring system 2 can illuminate multiple LEDs of different colors allowing for additional secondary or tertiary colors. In some embodiments, the patient monitoring system 2 may illuminate more than one LED. For example, the patient monitoring system 2 may illuminate both an amber LED and a green LED. Such illumination has the effect of creating a yellow state that may indicate a caution or a disabled indication. In some embodiments, the yellow indicator may appear on the patient support apparatus 20 and/or as well as projections on the floor. In some instances, disabling patient monitoring devices 16 may allow for safer operation of some ancillary equipment. When the equipment is removed and the interference issue is not a concern anymore, caregivers may be reminded by the yellow disabled indication of the patient monitoring system 2 that they may now enable the patient monitoring devices 16.

It should be understood that the approach of varying the colors of light indicators could be implemented to provide illumination in other applications. For example, various approaches to providing and using illuminating indication of different colors are disclosed in WO2016/196403, filed May 29, 2016, titled “PATIENT SUPPORT APPARATUS,” U.S. Pat. No. 1,117,289B2, filed Jul. 26, 2017, titled “PATIENT SUPPORT APPARATUS HAVING VITAL SIGNS MONITORING AND ALERTING,” U.S. Pat. No. 10,918,546B2, filed Jun. 10, 2020, titled “MULTI-ALERT LIGHTS FOR HOSPITAL BEDS,” AND U.S. Pat. No. 9,827,156B2, filed Nov. 12, 2012, titled “PERSON SUPPORT APPARATUS,” each of which is incorporated by reference herein for the disclosure of an illuminated bed condition. It should be understood that the approach of varying the colors of light indicators could be implemented to provide illumination these applications.

The present disclosure may also be embedded in a computer program product, which comprises all the features enabling the implementation of the methods described herein, and which when loaded in a computer system is able to carry out these methods. Computer program in the present context means any expression, in any language, code or notation, either statically or dynamically defined, of a set of instructions intended to cause a system having an information processing capability to perform a particular function either directly or after either or both of the following: a) conversion to another language, code or notation; b) reproduction in a different material form.

Although this disclosure refers to specific embodiments, the disclosure is not limited to the disclosed embodiments. It will be understood by those skilled in the art that various changes in form and detail can be made without departing from the subject matter set forth in the accompanying claims. For example, the disclosure has been illustrated and described in detail in the drawings and the foregoing description, such illustration and description are to be considered illustrative or exemplary and not restrictive. From reading the present disclosure, other modifications will be apparent to a person skilled in the art. Such modifications may involve other features, which are already known in the art and may be used instead of or in addition to features already described herein. In the claims, the word “comprising” does not exclude other elements or steps, and the indefinite article “a” or “an” does not exclude a plurality.

1. A patient monitoring system, comprising:
a patient support apparatus including an indicator, the indicator comprising a plurality of light sources,
a control system comprising a user interface (UI) including a display and a processor configured to change the display from a first display screen to a second display screen based on a first user input received on a first icon on the first display screen, and
wherein the first icon corresponds to a patient monitoring device configured to monitor a patient activity, wherein the second display screen is configured to indicate a first state of the patient monitoring device, wherein the indicator is configured to exhibit the first state by illuminating the plurality of light sources in a first condition, and wherein the first condition indicates the first state of the patient monitoring device.

2. The patient monitoring system of claim 1, wherein the control system is further configured to change the display from the second display screen to a third display screen based on a second user input received on a second icon on the second display screen, wherein the second icon corresponds to a second state of the patient monitoring device, different from the first state of the patient monitoring device.

3. The patient monitoring system of claim 2, wherein the control system is further configured to change the display from the third display screen to a fourth display screen based on a third user input received on a third icon on the third display screen, wherein the third icon corresponds to a disabled state of the patient monitoring device.

4. The patient monitoring system of claim 3, wherein the control system is further configured to change the display from the fourth display screen to a fifth display screen based on a fourth user input received on a fourth icon on the fourth display screen, wherein the fourth icon corresponds to a confirmation of the second state of the patient monitoring device.

5. The patient monitoring system of claim 4, wherein the control system is further configured to change the patient monitoring device from the first state to the second state if a user selects the fourth icon, and wherein the indicator is configured to exhibit a first color indicating the first state and a second color indicating the second state by illuminating one or more of the plurality of light sources.

6. The patient monitoring system of claim 1, wherein the plurality of light sources comprises a plurality of light emitting diodes (LEDs), and wherein each of the plurality of LEDs has a different color.

7. The patient monitoring system of claim 1, wherein the processor is further configured to control an intensity of light emitted from each of the plurality of light sources.

8. The patient monitoring system of claim 1, wherein the first state of the patient monitoring device is indicated by blend of light emitted from one or more of the plurality of light sources.

9. A control system, comprising:
a user interface (UI) including a display and a processor configured to change the display from a first display screen to a second display screen based on a first user input received on a first icon on the first display screen, and
wherein the first icon corresponds to a patient monitoring device configured to monitor a patient activity, wherein the second display screen is configured to indicate a first state of the patient monitoring device by illuminating an indicator comprising a plurality of light sources.

10. The control system of claim 9, wherein the control system is further configured to change the display from the second display screen to a third display screen based on a second user input received on a second icon on the second display screen, wherein the second icon corresponds to a second state of the patient monitoring device, different from the first state of the patient monitoring device.

11. The control system of claim 10, wherein the control system is further configured to change the display from the third display screen to a fourth display screen based on a third user input received on a third icon on the third display screen, wherein the third icon corresponds to a disabled state of the patient monitoring device.

12. The control system of claim 11, wherein the control system is further configured to change the display from the fourth display screen to a fifth display screen based on a fourth user input received on a fourth icon on the fourth display screen, wherein the fourth icon corresponds to a confirmation of the second state of the patient monitoring device.

13. The control system of claim 12, wherein the control system is further configured to change the patient monitoring device from the first state to the second state if a user selects the fourth icon, and wherein the control system is configured to illuminate the indicator to identify the second state of the patient monitoring device.

14. The control system of claim 13, wherein the first state and the second state are one of enabled and disabled.

15. The control system of claim 1, wherein the plurality of light sources comprises a plurality of light emitting diodes (LEDs), and each of the plurality of LEDs has a different color.

16. A patient monitoring system, comprising:
a patient support apparatus including an indicator,
a patient monitoring device configured to monitor a patient activity, and
wherein the indicator comprising a plurality of light sources is configured to exhibit a first color indicative of a first state of the patient monitoring device.

17. The patient monitoring system of claim 16, wherein the indicator is further configured to indicate a second state of the patient monitoring device by exhibiting a second color.

18. The patient monitoring system of claim 17, wherein the plurality of light sources comprises a plurality of light emitting diodes (LEDs), and each of the plurality of LEDs has a different color.

19. The patient monitoring system of claim 16, wherein a control system is configured to control an intensity of light emitted from each of the plurality of light sources.

20. The patient monitoring system of claim 16, wherein the first state of the patient monitoring device is indicated by blend of light emitted from one or more of the plurality of light sources.
